(19) United States
(12) Patent Application Publication
(10) Pub. No.: US 2009/0090376 A1
(43) Pub. Date: Apr. 9, 2009

(54) NAIL-CLEANING TOOL

(75) Inventor: Ching-Yu WANG, Shih Ting Hsiang (TW)

Correspondence Address: PATENTTM.US, P. O. BOX 82788, PORTLAND, OR, 97282-0788, US

(73) Assignee: RESIN ENTERPRISE CO., LTD., Taipei Hsien (TW)

(21) Appl. No.: 11/866,930

(22) Filed: Oct. 3, 2007

(51) Int. Cl. A45D 29/00; A61M 35/00

(52) U.S. Cl. 132/75.3; 604/1

(57) ABSTRACT

A nail-cleaning tool has a rod, a removing end, a scraping end and a sheath. The scraping end is spatula-shaped. The sheath is an absorbent material mounted over the removing end. Therefore, users can use the removing end to absorb nail polish remover then clean a surface of the nail during removal and application. The users can further use the scraping end to scrap an edge of each nail adjacent to skin. Therefore, the nail can be cleaned thoroughly while minimizing waste and reducing direct skin contact with the nail polish remover.

### BACKGROUND OF THE INVENTION

1. Field of Invention

The present invention relates to a nail-cleaning tool, and more particularly to a nail-cleaning tool that cleans nails conveniently and thoroughly.

2. Description of the Related Art

People have hands and feet with nails. The nails have an edge and are formed on fingers and toes of the people and have skin adjacent to each nail.

Many people, especially female, apply nail polish to their nails to improve appearance of their nails, by adding color for fashion, to coordinate clothing or the like and also for enjoyment. Nail art has become very popular in recent years wherein the addition of patterns, adhered sticks, glass stones or other 3D accessories are adhered to the nails.

Generally, people use cotton pads with a nail polish remover being a solvent, frequently comprising acetone, to remove the nail polish. The nail polish on a surface of each nail is easily removed. However, each cotton pad has a rectangular or square shape and the solvent in the nail polish remover easily contacts the skin adjacent to the nails. Additionally, the edge of each nail adjacent to the skin is difficult to remove using cotton pads. Therefore, the nail polish cannot be thoroughly removed quickly, which annoys people and wastes their time.

Finally much of the cotton pad is not utilized for cleaning, but is merely to provide a surface for gripping therefore, much material is wasted and thrown away.

To overcome the shortcomings, the present invention provides a nail-cleaning tool to mitigate or obviate the aforementioned issues.

### SUMMARY OF THE INVENTION

The primary objective of the present invention is to provide a nail-cleaning tool that cleans nails conveniently and thoroughly.

To achieve the objective, a nail-cleaning tool in accordance with the present invention comprises a rod, a removing end, a scraping end and a sheath. The scraping end is spatula-shaped. The sheath is an absorbent material and is mounted over the removing end. Therefore, users can use the removing end to soak a nail polish remover then clean a surface of each nail during removal and application. The users can further use the scraping end to scrap an edge of each nail adjacent to skin. Therefore, the nail can be cleaned thoroughly while minimizing waste and reducing direct skin contact with the nail polish remover.

Other objectives, advantages and novel features of the invention will become more apparent from the following detailed description when taken in conjunction with the accompanying drawings.

### BRIEF DESCRIPTION OF THE DRAWINGS

FIG. 1 is a perspective view of a nail-cleaning tool in accordance with the present invention;

FIG. 2 is an operational perspective view of the nail-cleaning tool in FIG. 1, shown with a removing end being utilized;

FIG. 3 is an operational perspective view of the nail-cleaning tool in FIG. 1 shown with a scraping end being utilized; and

FIG. 4 is a perspective view of the nail-cleaning tool in FIG. 1, shown with a storage device.

### DETAILED DESCRIPTION OF THE INVENTION

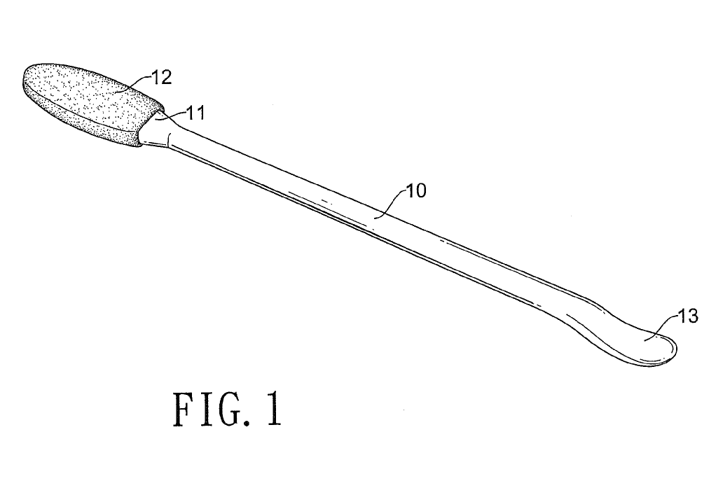

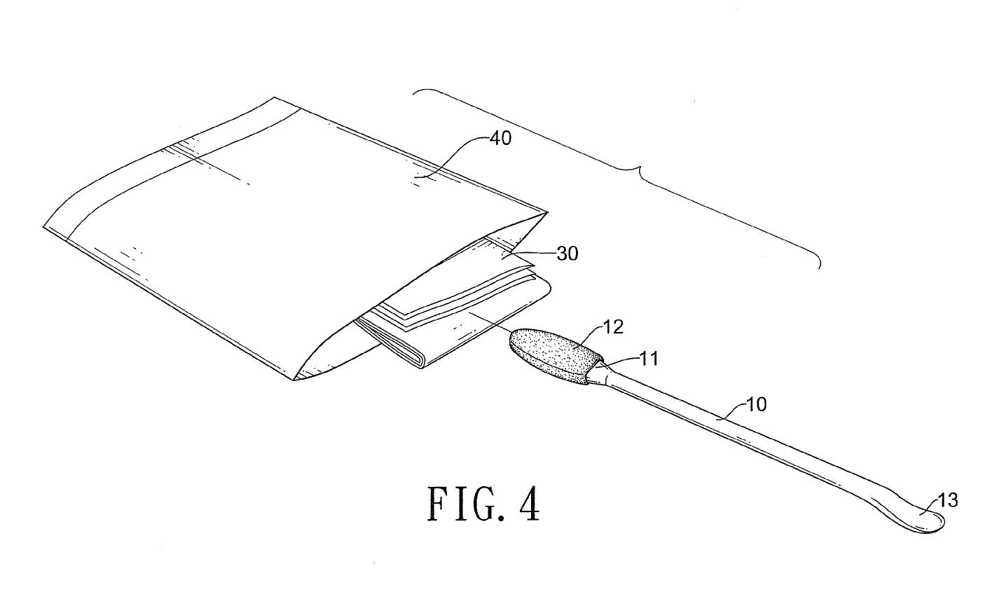

With reference to FIGS. 1 and 4, a nail-cleaning tool in accordance with the present invention comprises a rod (10), a removing end (11), a scraping end (13), a sheath (12) and may be further implemented with a storage device (40).

The rod (10) may be plastic, wood, paper or the like.

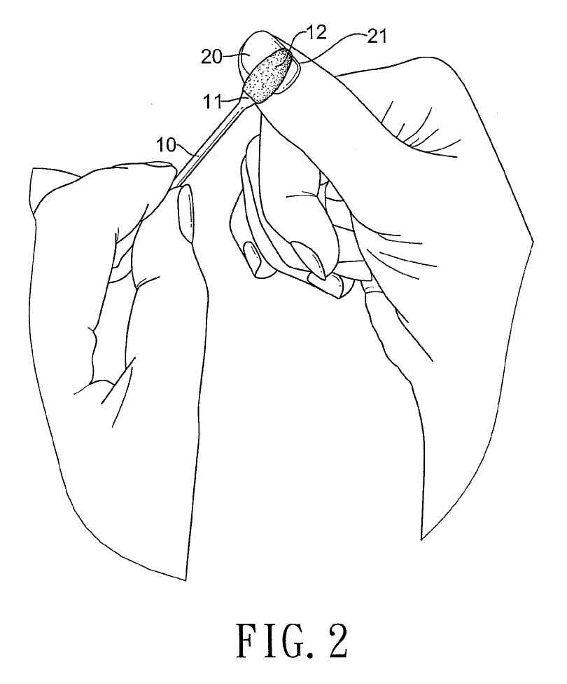

With further reference to FIG. 2, the removing end (11) is a flattened tongue shape and has an outer surface.

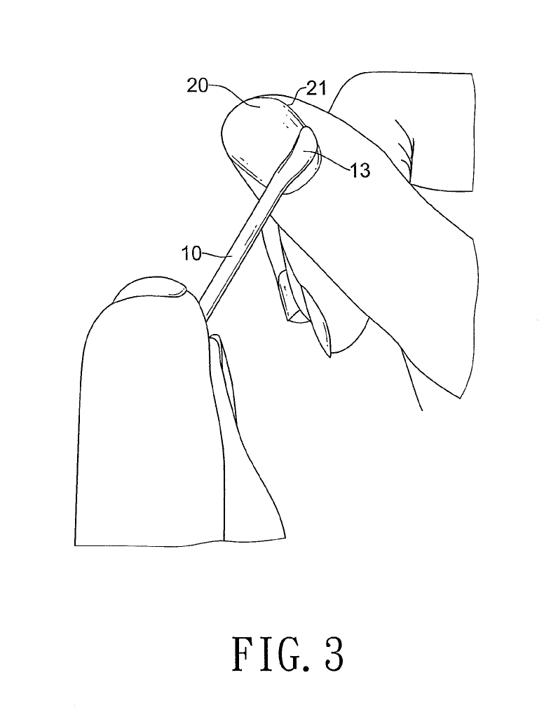

With further reference to FIG. 3, the scraping end (13) is spatula shaped and is used to scrap an edge (21) of the nail (20) adjacent to skin to clean the nail (20) thoroughly.

The sheath (12) is an absorbent material mounted and may be replaceably mounted over the outer surface of the removing end (11). The absorbent material may be cotton, rayon, nonwovens or the like, wherein nonwovens are materials that are neither knitted or woven and include felt and the like, to the absorbent material can absorb nail polish remover or nail polish. The sheath (12) may be mounted securely on the rod (10). Therefore, the nail cleaning tool may be implemented as a disposable item, replaced after each use.

The storage device (40) may be a bag, box or the like and may further contain a tissue or a cotton pad (30). The tissue or cotton pad (30) packs the sheath (12) to keep the sheath (12) clean before using.

Because the sheath (12) is held by the rod (10), the sheath (12) is smaller than the nail (20) and wastes less material than prior art since no material is required to grasp the sheath (12). Moreover, this prevents direct skin contact with the nail polish remover. Users can also hold the rod (10) and use the removing end (11) to absorb nail polish remover before using to clean a surface of each nail (20) thereby minimizing skin contact with the nail polish remover. Because the removing end (11) is flat, the removing end corresponds to the surface of the nail (11) and cleans the surface of the nail (11) easily. Additionally, when the users apply nail polish, the removing end (11) can be used to soak up excess nail polish, especially that is depositing on the edge (21) of the nail (20) adjacent to the skin to improve appearance of the nail polish on the nail and prevent the nail polish from permeating and irritating the skin.

The ends (11, 13) of the nail-cleaning tool (10) have two different functions including cleaning the surface of the nail (20) and the edge (21) of the nail (20) adjacent to the skin. Therefore, the users can use the nail-cleaning tool (10) to thoroughly clean the nail (20).

Even though numerous characteristics and advantages of the present invention have been set forth in the foregoing description, together with details of the structure and function of the invention, the disclosure is illustrative only. Changes may be made in detail, especially in matters of shape, size and arrangement of parts within the principles of the invention to the full extent indicated by the broad general meaning of the terms in which the appended claims are expressed.

What is claimed is:

1. A nail-cleaning tool comprising
a rod;
a removing end;
a scraping end being spatula shaped; and
a sheath being an absorbent material mounted over the removing end.

2. The nail-cleaning tool as claimed in claim 1, wherein the removing end is a flattened tongue shape.

3. The nail-cleaning tool as claimed in claim 1, wherein the absorbent material is cotton.

4. The nail-cleaning tool as claimed in claim 2, wherein the absorbent material is cotton.

5. The nail-cleaning tool as claimed in claim 1, wherein the absorbent material is rayon.

6. The nail-cleaning tool as claimed in claim 2, wherein the absorbent material is rayon.

7. The nail-cleaning tool as claimed in claim 1, wherein the absorbent material is nonwovens.

8. The nail-cleaning tool as claimed in claim 2, wherein the absorbent material is nonwovens.
